(19) United States
(12) Patent Application Publication
(10) Pub. No.: US 2008/0146659 A1
(43) Pub. Date: Jun. 19, 2008

(54) Hypoglycemic agent, hepatoprotecting agent and anticancer agent containing lignans derived from hongdoushan

(75) Inventors: Shigetoshi Kadota, Toyama (JP); Takahiro Nobukawa, Kanagawa (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: KOTOSUGI INC., Sagamihara-shi (JP)

(21) Appl. No.: 12/068,590

(22) Filed: Feb. 8, 2008

Related U.S. Application Data
(62) Division of application No. 10/522,186, filed on Jan. 24, 2005.

(30) Foreign Application Priority Data
Jul. 24, 2002 (JP) JP2002-214694
Apr. 24, 2003 (JP) JP2003-119178

(51) Int. Cl. A61K 31/341; A61P 3/08

(52) U.S. Cl. 514/461

(57) ABSTRACT

Drugs containing Taxiresinol, (7′R)-7′-Hydroxylariciresinol, Secoisolariciresinol and Isotaxiresinol, which are lignans contained in Hongdoushan, as the active ingredients. Drugs containing an extract, which is obtained by extracting a Hongdoushan plant with water and further extracting the obtained extract with an organic solvent, as the active ingredient. These drugs are useful particularly as a hypoglycemic agent, a liver protecting agent and an anticancer agent.

This application is a divisional application of U.S. Ser. No. 10/522,186, filed on Jan. 24, 2005, which is a National Stage of PCT/JP200.3/09370, filed Jul. 23, 2003, and which claims priority of Japanese Patent Application Nos. 2002-214694 and 2003-119178 filed on Jul. 24, 2002 and Apr. 24, 2003, respectively, which are hereby incorporated by reference in their entirety.

### TECHNICAL FIELD

The present invention relates to a drug containing compounds of lignans and more particularly to a hypoglycemic agent, a hepatoprotective agent, and an anticancer agent which contain the above-mentioned compounds.

### BACKGROUND ART

Diabetes mellitus is a group of metabolic disorders relating to carbohydrate, lipid, and protein. It has been reported that the disease affects approximately 10 percent of the world's population. We have, however, now no effective medicine for prevention and remedy of the disease other than insulin and other hypoglycemic agents which may have many kinds of side effects.

The liver is called a “silent organ” because it has strong natural healing power and shows few clear symptoms until disorders develop to some degree. Liver has functions of the essence in maintaining human life, such as metabolism, glycemic control, detoxification, bile circulation control, and nutrient storage. Disturbance of liver function can be caused by many kinds of etiology and shows many different disease states. Despite such diversification, there is a real need for drugs for treatment of chronic, active hepatitis in the medical care field. For such need, antiviral drugs, immunoregulatory drugs and other drugs for casual therapy as well as hepatoprotective drugs have been under development.

As for anticancer drugs, approximately 60 drugs have been on the market and a further approximately 40 are under clinical testing; nevertheless, cancer is one of the major causes of death. This fact requires immediate development of new drugs for cancer.

Hongdoushan (nomenclature: Taxus yunnanensis), a evergreen tree which grows on the high-mountain area in Yunan, China, is known as an medicinal plant effective for anti-inflammation, diuresis, lowering blood pressure, decreasing lipids in the blood and the like. The wood of hongdoushan has also been proven to contain paclitaxel (Taxol), which is an anticancer agent.

A tree tea derived from a crushed and grinded trunk of hongdoushan is disclosed in Japanese Patent Laid-Open No. 10-120582, in which an attention is focused on medicinal benefits of hongdoushan. Japanese Patent Laid-Open Nos. 2000-236835 and 2000-236836 disclose a food derived from a mixture of a crushed and grinded trunk of hongdoushan and specific medical plants.

The recent permission of limited export of hongdoushan to Japan and the United States of America by the Chinese government has promoted studies of ingredients contained in hongdoushan and pharmacologic actions thereof.

An object of the present invention is to provide new medicinal applications of ingredients contained in hongdoushan on the basis of their biological activities newly discovered by the present inventors. A further object of the present invention is to provide new medicinal applications of fractions extracted from hongdoushan.

### BRIEF DISCLOSURE OF THE INVENTION

The present inventors have found that compounds of lignans contained in hongdoushan are biologically active in vitro and in vivo, and the present invention has been accomplished on the basis of this discovery.

The present invention relates to a drug comprising a compound shown in the formula (1) (wherein R.sup.1 is a hydrogen or a hydroxyl group, R.sup.2 is either an alkyloxy group or a hydroxyl group, both with the carbon number of 1 to 4, and R.sup.3 is an alkyloxy group with the carbon number of 1 to 4) or a pharmaceutically acceptable salt or ester of said compound in the formula (1) as an effective ingredient.

The present invention also relates to a drug comprising a compound shown in the formula (2) (wherein R.sup.4 and R.sup.5 are an alkyloxy group with the carbon number of 1 to 4) or a pharmaceutically acceptable salt or ester of said compound in the formula (2) as an effective ingredient.

Furthermore, the present invention relates to a drug comprising a compound shown in the formula (3) (wherein R.sup.6 is an alkyloxy group with the carbon number of 1 to 4) or a pharmaceutically acceptable salt or ester of said compound in the formula (3) as an effective ingredient.

In an embodiment of the present invention, a drug according to claim 1 is a hypoglycemic agent, a hepatoprotective agent or an anticancer agent.

In another embodiment of the present invention, a drug is a hypoglycemic agent, a hepatoprotective agent or an anticancer agent.

In still another embodiment of the present invention, a drug is a hypoglycemic agent, a hepatoprotective agent or an anticancer agent.

In the present invention, ester means a compound in which a hydroxyl group of a methylol group (CH.sub.20H) in the formula (1), (2) and (3) combines with an organic acid or an inorganic acid, and a water molecule is removed. In the formula (2) or (3), one of the two methylol groups in one molecule may be esterified or both of them may be esterified. Any of the pharmaceutically acceptable esters publicly known in the medical and pharmaceutical fields may be used without restriction. For example, acetic acid may be used as an organic acid, and phosphoric acid may be used as an inorganic acid.

Any of the salts induced from an inorganic or organic base is acceptable, including a salt in which a phenol group in a compound becomes a phenoxide ion group and/or a salt in which a methylol group becomes a methyloxide ion group. Any of the pharmaceutically acceptable salts publicly known in the medical and pharmaceutical fields may be used without restriction. Such salts include alkaline metal, alkaline-earth metal, and amine salt.

Furthermore, the present invention relates to a drug having as an effective ingredient an extract which is extracted with an organic solvent from an aqueous extract which is extracted with water from the wood of hongdoushan.

In a preferred embodiment of the present invention, a drug is a hypoglycemic agent, a hepatoprotective agent or an anticancer agent.

In the present invention, a hypoglycemic agent means a drug used for prevention and treatment of diabetes, a hepatoprotective agent means a drug for recovery and preservation of liver function, and an anticancer agent means a drug used for treatment, prevention and recurrence prevention of cancer.

In the compound of the formula (1), when R.sup.1 is H, R.sup.2 is OH, and R.sup.3 is CH.sub.3O, a compound of the resulting formula, i.e., the formula (4), is Taxiresinol (hereinafter called “TAX”).

In the compound of the formula (1), when R.sup.1 is OH, R.sup.2 is CH.sub.30, and R.sup.3 is CH.sub.30, a compound of the resulting formula, i.e., the formula (5), is (7′R)-7′-Hydroxylariciresinol (hereinafter called “HYL”).

In the compound of the formula (2), when R.sup.4 is CH.sub.30 and R.sup.5 is CH.sub.30, a compound of the resulting formula, i.e., the formula (6), is Secoisolariciresinol (hereinafter called “SIL”).

In the compound of the formula (3), when R.sup.6 is CH.sub.30, a compound of the resulting formula, i.e., the formula (7), is Isotaxiresinol (hereinafter called “ITX”).

TAX, HYL, SIL, and ITX, which are contained in the wood (leaves, bark, body, core, root and the like) of hongdoushan, can be extracted and isolated in the following steps. First, an aqueous extract is extracted with heated water from the wood. Second, an organic solvent fraction is extracted with an organic solvent (e.g., ethyl acetate) from the aqueous extract. Third, these compounds are isolated from the organic solvent fraction by way of chromatography (column chromatography, thin-layer chromatography, HPLC and the like).

The compound having the formula (1), (2) or (3) can be synthesized from the compounds obtained in the above steps, by means of organic synthesis.

The methoxy group (CH.sub.30) of TAX may be substituted by an ethoxy group (C.sub.2H.sub.50), a propyloxy group (C.sub.3H.sub.70) or a butyloxy group (C.sub.4H.sub.90). Each of the two methoxy groups of HYL may be substituted by an ethoxy group, a propyloxy group or a butyloxy group, and two alkyloxy groups may be either the same or different. Each of the two methoxy groups of SIL may be substituted by an ethoxy group, a propyloxy group or a butyloxy group, and two alkyloxy group may be either the same or different. A methoxy group of ITX may be substituted by an ethoxy group, a propyloxy group or a butyloxy group.

In order to obtain an organic solvent extract of an aqueous extract of hongdoushan of the present invention, an aqueous solution is extracted with water from the wood (leaves, bark, body, core, root and the like) of hongdoushan as a first step. The body and bark (collectively “xylem”) of the wood are preferable. Preferably, the extraction is carried out with heated water. In a more specific embodiment of the extraction operation, for instance, the crushed and grinded wood and 2 to 20 times its volume of purified water (e.g., 1 kg of crushed and grinded wood and 2 to 20 L of purified water) are mixed, and the extraction is carried out at room temperature or with heating, preferably with heating, more preferably at 100.degree.C. for 1 minute to 2 hours, preferably for 20 minutes to 1 hour. Then, a supernatant is collected by means of filtration or centrifugation.

The second step is to obtain an organic solvent solution by means of extraction with an organic solvent from the aqueous solution obtained in the first step. The volume of the aqueous solution may be reduced before the extraction by means of vacuum condensation and so on. The extraction may be carried out with an organic solvent after an inorganic salt is dissolved in the aqueous solution. As the organic solvent, any organic solvent commonly used for extraction of a compound from water solution, such as ethyl acetate, alcohols, ethers, aliphatic hydrocarbons, aromatic hydrocarbons (benzene, toluene and the like), and pyridine, may be used. Preferable are polar organic solvents, including those having an oxygen atom and a nitrogen atom within their molecules. Most preferable are ethyl acetate and diethyl ether.

Next, the organic solvent is removed from the organic solvent solution in accordance with the usual method to finally obtain the organic solvent extract.

Drugs of the present invention may be administered orally, parenterally or subcutaneously. Any administration method commonly used for drugs, such as tablet, coated tablet, capsule, solution, syrup, powder and suppository, may be used without restriction.

Tablets may be manufactured by mixing a compound(s) or an extract(s) with a vehicle (lactose, glucose, sucrose, mannitol and the like), a disintegrating agent (cornstarch, alginic acid and the like), a binder (starch, gelatin and the like), a lubricant (magnesium stearate, talc and the like) and/or delayed-release agents (carboxymethyl cellulose, cellulose acetate phthalate, polyvinyl alcohol and the like). Tablets with one or more layers are acceptable.

Coated tablets can be manufactured by coating a core manufactured in the same way as tablets are done with materials commonly used for tablet coating, such as collidone, shellac, gum Arabic, talc, titanium dioxide, and sucrose. Coated tablets with one or more layers are acceptable. The vehicles described above may be used.

Solutions and syrups can be fabricated by appropriately adding water, saccarides (erythritol, xylitol, mannitol, sucrose, trehalose, maltose, fructose, sorbit, honey and the like), antiseptic agents (paraben and the like), aroma chemicals, coloring agents, and oils (soybean oil and the like) to a compound(s) of lignans and mixing them.

Capsules containing a drug of the present invention can be manufactured by encapsulating a compound(s) or an extract(s) with a gelatin capsule or by mixing a compound(s) or an extract(s) with an inactive support(s) such as lactose and sorbitol and then encapsulating the mixture with a gelatin capsule or wrapping it with a gelatin film.

A dose of a compound(s) or extract(s) of the present invention is commonly 1 to 1,000 mg/person/day. However, an appropriate dose is intended to be decided by first administering a small amount of the compound(s) or extract(s) and then increasing the dose until the intended effect is obtained.

The lignans of the present invention are compounds of hongdoushan, a medicinal plant that has been safely ingested, and are safe substances.

The present invention provides new drugs useful for treatment of illnesses and health promotion, in particular, useful drugs as hypoglycemic agent, hepatoprotective agent, and anticancer agent.

### BRIEF DESCRIPTION OF THE DRAWINGS

FIG. 1 is an illustration showing an extraction and fractionation procedure of ingredients from hongdoushan xylem.

FIG. 2 is a graph showing the results of tests for effects of SIL on decrease in blood glucose level using rats.

FIG. 3 is a graph showing the results of tests for effects of ITX on decrease in blood glucose level using rats.

FIG. 4 is a graph showing the results of measurements of transaminase in blood serum of mice which were given TAX and HYL.

FIG. 5 is a graph showing the results of measurements of transaminase in blood serum of mice which were given SIL and ITX.

FIG. 6 is a graph showing the results of measurements of transaminase in blood serum of mice which were given an ethyl acetate soluble fraction.

FIG. 7 is a graph showing the results of measurements of TNF-.alpha. in blood serum of mice which were given TAX and HYL.

FIG. 8 is a graph showing the results of measurements of TNF-.alpha. in blood serum of mice which were given SIL and ITX.

FIG. 9 is a graph showing the results of measurements of inhibitory activity of TAX and HYL on cell death in primary cultured mouse hepatocytes.

FIG. 10 is a graph showing the results of measurements of inhibitory activity of SIL and ITX on cell death in primary cultured mouse hepatocytes.

### EXPLANATION OF CODES

TAX: Taxiresinol [0050] HYL: (7′R)-7′-Hydroxylariciresinol [0051] SIL: Secoisolariciresinol [0052] ITX: Isotaxiresinol [0053] SI: Silymarin

### MOST PREFERRED EMBODIMENT TO CARRY OUT THE INVENTION

The present invention is explained in more detail by the following embodiment. Materials, extraction methods of compounds and the like described in the embodiment of the present invention are merely examples and are not intended to restrict the scope of the present invention thereto.

### (Isolation)

A body and bark (collectively “xylem”) of hongdoushan was grinded by a grinder to obtain powder of 30 mesh pass. The powder was dried. The dried powder (850 g) was extracted with purified water (4 L) under reflux for 45 minutes. A residue remaining after filtration was further extracted with purified water (4 L) under reflux for 45 minutes. Furthermore, the same extraction operation was carried out once again. Aqueous solution layers obtained after the three extractions were collected and vacuum concentrated to obtain 52.5 g of aqueous extract.

Next, the aqueous extract (52.5 g) was extracted with ethyl acetate (500 mL), and an ethyl acetate layer was separated. A residue remaining after the separation was further extracted with ethyl acetate (500 mL). Furthermore, the same extraction operation was carried out once again. Ethyl acetate layers obtained after the three extractions were collected and then vacuum concentrated to obtain 31.1 g of ethyl acetate soluble fraction.

The ethyl acetate soluble fraction (34.1 g) was applied on a silica gel column (inner diameter 3.5 cm, length 60 cm, packing materials: Silica gel 60 (Nacalai Tesque, Inc.)) and eluted with a solvent of methanol and chloroform to obtain 9 fractions of each 500 mL. Table 1 shows the composition of the solvent, the weight of the eluate obtained after the vacuum concentration of each fraction, and the components contained in each fraction. TABLE-US-00001 TABLE 1 Column chromatography of ethyl acetate soluble fractions of aqueous extract of hongdoushan Composition of Fraction Solvent (*1) Weight Number MeOH % (g.) Components 1 0 0.31 2 0 0.30 3 1 0.30 4 1-5 (*2) 2.78 SIL 5 5 1.68 SIL, TAX, HYL 6 10 12.5 7 10-20 (*3) 7.84 ITX 8 20 1.41 9 30 1.00 (*1) The solvent is a mixture of chloroform and methanol. The figures in the list show the ratio of the methanol to the mixture in percentage. (*2) The fraction mixed with the eluate at 1%:100 mL, 2%:100 mL, 3%:100 mL, 4%:100 mL, and 5%:100 mL. (*3) The fraction mixed with the eluate at 12%:100 mL, 14%:100 mL, 16%:100 mL, 18%: 100 mL, and 20%:100 mL.

Crystallized SIL (840 mg) was obtained after vacuum concentration of the eluate of fraction 5. The residue was separated using a preparative thin-layer chromatography. A thin-layer plate used was a Kieselgel 60 F 254 thickness 0.5 mm (manufactured by Merck), and a developing solvent used was methanol:chloroform/10:90 solution. Rf values were TAX:0.25 and HYL:0.21. Rf value of SIL under the same condition was 0.36. TAX (38.9 mg) and HYL (10.2 mg) were obtained by the preparation.

Structures of TAX, HYL, SIL, and ITX were identified and confirmed on the basis of spectral and chemical analyses. The major analytical data are given below:

### TAX(Taxiresinol):Light Brown Amorphous Solid

.sup.1H NMR (CD.sub.3OD) delta.6.76 (1H, d, J=2.0, Hz, H-2), 6.76 (1H, d, J=2.0 Hz, H-2′), 6.71 (1H, d, J=8.0 Hz, H-5), 6.69 (1H, d, J=8.0 Hz, H 5′), 6.63 (1H, dd, J=2.0, 8.0 Hz, H-6′), 6.61 (1H, dd, J=2.0, 8.0 Hz, H-6), 4.66 (1H, d, J=6.9 Hz, H-7′), 3.93 (1H, dd, J=6.4, 8.3 Hz, H-9′), 3.80 (3H, s, H—OMe), 3.78 (1H, dd, J=8.0, 10.5 Hz, H-9′), 3.68 (1H, dd, J=5.9, 8.3 Hz, H-9′), 3.60 (1H, dd, J=6.4, 10.5 Hz, H-9′), 2.90 (1H, dd, J=4.6, 13.4 Hz., H-7), 2.70 (1H, m, H-8), 2.45 (1H, dd, J=11.5, 13.4 Hz, H-7), 2.35 (1H, m, H-8′);

.sup.13C NMR (CD.sub.3OD) delta.148.9 (C-3′), 146.3 (C-3), 145.7 (C-4′), 145.7 (C-4), 135.8 (C-1′), 133.5 (C-1), 122.1 (C-6), 118.6 (C-6′), 116.1 (C-5), 116.1 (C-5′), 114.1 (C-2′), 113.4 (C-2), 83.9 (C-7′), 73.4 (C-9), 60.4 (C-9′), 56.3 (C—OMe), 54.0 (C-8′), 43.8 (C-8), 33.6 (C-7).

[.alpha.].sub.D.sup.25+23.degree. (c=0.32 in Ethanol)

### HYL ((7′R)-7′-Hydroxylariciresinol):Colorless Amorphous Solid

.sup.1H NMR (CD.sub.3OD).delta.6.91 (1H, d, J=2.0 Hz, H-2), 6.86 (1H, 2, H-2′), 6.79 (1H, dd, J=8.0, 2.0 Hz, H-6), 6.74 (1H, d, J=8.0 Hz, H-5), 6.73 (2H, m, H-5′ and H-6′), 4.61 (1H, d, J=7.3 Hz, H-7), 4.47 (1H, d, J=8.6 Hz, H-7′), 4.23 (1H, dd, J=9.0, 4.4 Hz, H-9′), 3.93 (1H, dd, J=9.0, 7.8 Hz, H-9′), 3.84 (3H, s, H—OMe), 3.82 (3H, s, H—OMe), 3.21 (1H, dd, J=10.9, 5.9 Hz, H-9),3.30 (1H, dd, J=10.9, 4.6 Hz, H-9),2.54 (1H, m, H-8′), 1.88 (1H, m, H-8);

.sup.13C NMR (CD.sub.3OD) delta.148.9 (C-3), 148.9 (C-3′), 147.1 (C-4), 147.1 (C-4′), 136.1 (C-1′), 134.7 (C-1), 120.7 (C-6′), 120.2 (C-6), 115.9 (C-5′), 115.9 (C-5), 111.5 (C-2′), 111.0 (C-2), 85.0 (C-7), 76.6 (C-7′), 71.4 (C-9′), 62.2 (C-9), 56.4 (C—OMe), 56.3 (C—OMe), 53.3 (C-8), 50.7 (C-8′).

[.alpha.].sub.D.sup.25+4.6.degree. (c=0.18 in Methanol)

### SIL (Secoisolariciresinol):Colorless Crystal

.sup.1H NMR (CD.sub.3OD), .delta.6.66 (2H, d, J=8.0 Hz, H-5), 6.58 (2H, d, J=2.0 Hz, H-2), 6.53 (2H, dd, J=2.0, 8.0 Hz, H-6), 3.71 (3H, s, H—OMe); 3.53 (4H, d, J=4.3 Hz, H-9), 2.56 (2H, dd, J=7.3, 13.7 Hz, H-7), 2.52 (2H, dd, J=7.7, 13.7 Hz, H-7), 1.88 (2H, m, H-8);

.sup.13C NMR (CD.sub.3OD).delta.148.6 (C-3), 145.3 (C-4), 133.7 (C-1), 122.6 (C-6), 113.3 (C-2), 115.7 (C-5), 61.9 (C-9), 56.1 (C—OMe), 44.0 (C-8), 36.0 (C-7)

[.alpha.].sub.D.sup.25-32.0.degree. (c=0.1 in Acetone)

### ITX(Isotaxiresinol):Colorless Amorphous Solid

.sup.1H NMR (CD.sub.3OD), .delta.6.69 (1H, d, J=8.0 Hz, H-5′), 6.61 (1H, s, H-5), 6.52 (1H, d, J=2.0 Hz, H-2′), 6.50 (1H, dd, J=2.0, 8.0 Hz, H-6′), 6.19 (1H, s, H-2), 4.67 (2H, m, H-9), 4.67 (1H, m, H-9′), 4.66 (1H, d, J=6.9 Hz, H-7′), 3.77 (3H, s, H—OMe), 3.40 (1H, dd, J=4.3, 11.1 Hz, H-9′), 2.73 (1H, br d, J=6.8 Hz, H-7), 1.97 (1H, m, H-8), 1.71 (1H, m, H-8′)

.sup.13C NMR (CD.sub.3OD) delta.147.1 (C-3), 146.2 (C-3′), 145.2 (C-4), 144.6 (C-4′), 138.7 (C-1′), 134.3 (C-1), 128.9 (C-6), 122.0 (C-6′), 117.4 (C-2), 117.3 (C-2′), 116.1 (C-5′), 112.3 (C-5), 66.0 (C-9), 62.4 (C-9′), 56.4 (C—OMe), 48.1 (C-8′), 47.8 (C-7′), 40.1 (C-8), 33.5 (C-7)

[.alpha.].sub.D.sup.25+47.3.degree. (c=0.4 in Ethanol)

The identified structures of TAX and SIL were found to be identical to those described in Mujumdar, R. B.; Srinivasan, R. & Venkataraman, K., Taxiresinol, A New Lignan in the Heartwood of Taxus baccata; Indian J. Chem., 40, 677-680 (1972). The identified structure of HYL was found to be identical to that described in Barrero, A. F.; Haidour, A.; Dorado, M. M.; Gravalos, D. & Quesada, T. G., Lignans from the wood of Abies pinsapo; J. Nat. Prod., 57, 713-719 (1994). The identified structure of ITX was found to be identical to that described in King, F. E.; L. Jurd & King, T. J., isoTaxiresinol (3′-Dimethylisolariciresinol), A New Lignan extracted from the Heartwood of the English Yew, Taxus baccata; J. Chem. Soc., 17-24 (1952).

### (Extraction and Fraction)

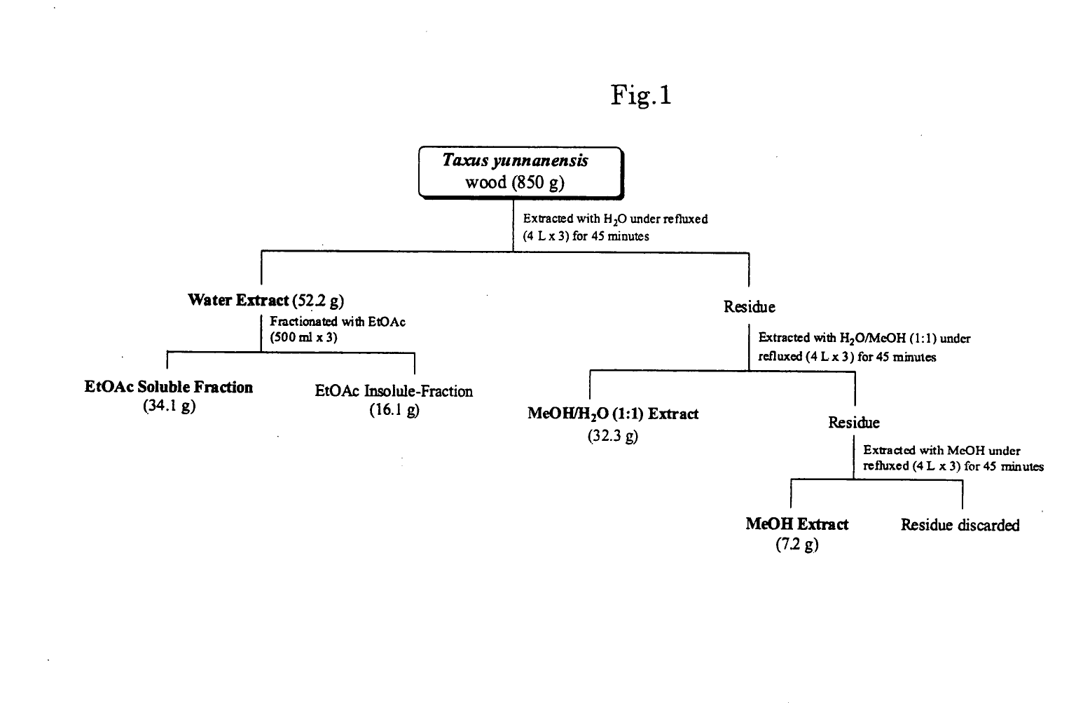

Referring to FIG. 1, an extraction and fraction operation of ingredients from hongdoushan xylem is illustrated.

Dried xylem powder of hongdoushan (850 g and 30 mesh pass) was extracted with purified water (4 L) under reflux for 45 minutes. The residue remaining after filtration was further extracted with purified water (4 L) under reflux for 45 minutes. Furthermore, the same extraction operation was carried out once again. Aqueous solution obtained layers after the three extractions were collected and vacuum concentrated to obtain 52.5 g of aqueous extract.

Next, the aqueous extract (52.5 g) was extracted with ethyl acetate (500 mL), and an ethyl acetate layer was isolated. A residue remaining after the isolation was further extracted with ethyl acetate (500 mL). Furthermore, the same extraction operation was carried out once again. Ethyl acetate layers obtained after the three extractions were collected and vacuum concentrated to obtain 34.1 g of ethyl acetate soluble fraction.

The residue, the aqueous solution remaining after the above-mentioned extraction, was vacuum concentrated to obtain 16.1 g of ethyl acetate insoluble fraction.

The residue (xylem powder) remaining after the extraction of said aqueous extract was extracted with a mixture (4 L) of methanol and water (1 to 1) under reflux for 45 minutes. After filtration, the same extraction operation was carried out 2 times. Solutions after the three extractions were vacuum concentrated to obtain 32.3 g of methanol/aqueous extract.

Next, a residue (xylem powder) was extracted under reflux with methanol (4 L) for 45 minutes. After filtration, the same extraction operation was carried out 2 times. Solutions after the three extractions were collected and vacuum concentrated to obtain 7.2 g of methanol extract.

### Example 1

### Decrease Activity in Blood Glucose Level

Activities of compounds of lignans and hongdoushan fractions in decreasing blood glucose level were assessed by using rats.

Blood serums were obtained after the blood cell separation of the whole blood samples collected from the rats. Measurement of the blood glucose level was carried out by using the blood serum, a Glucose CII-TEST WAKO (manufactured by Wako Pure Chemical Industries, Ltd.) as a reagent and a UV-160A (manufactured by Shimadzu Corporation) as an instrument for absorbance measurement.

Male rats of Wistar strains (age: 5-6 weeks, weight: 180-200 g) kept fast for 16 hours. Then, citrate buffer solution (pH 4.2) of streptozocin (hereinafter called “STZ”) was injected into the rats' abdominal cavity in a dose of 55 mg/kg (weight of rat). Blood samples were taken from the tail vein of the rats 5 days after the injection, to measure blood glucose level. Rats with blood glucose level higher than 250 mg/dL were considered to be diabetic and were used for the tests.

Compounds or fractions were injected into the abdominal cavity of the diabetic rats in a dose of 10 mg/kg (weight of rat) 5 times at intervals of 12 hours. Blood samples were taken from the tail veins of the rats to measure the blood glucose level. Averages and standard deviations of measured values were calculated for groups, each of which had 4 rats. The groups included a negative control group, which was given normal saline solution, and a positive control group of said diabetic rats, which were given a mixture of tolbutamide in a dose of 200 mg/kg (weight of rat) and buformin in a dose of 1 mg/kg (weight of rat) through their abdominal cavity.

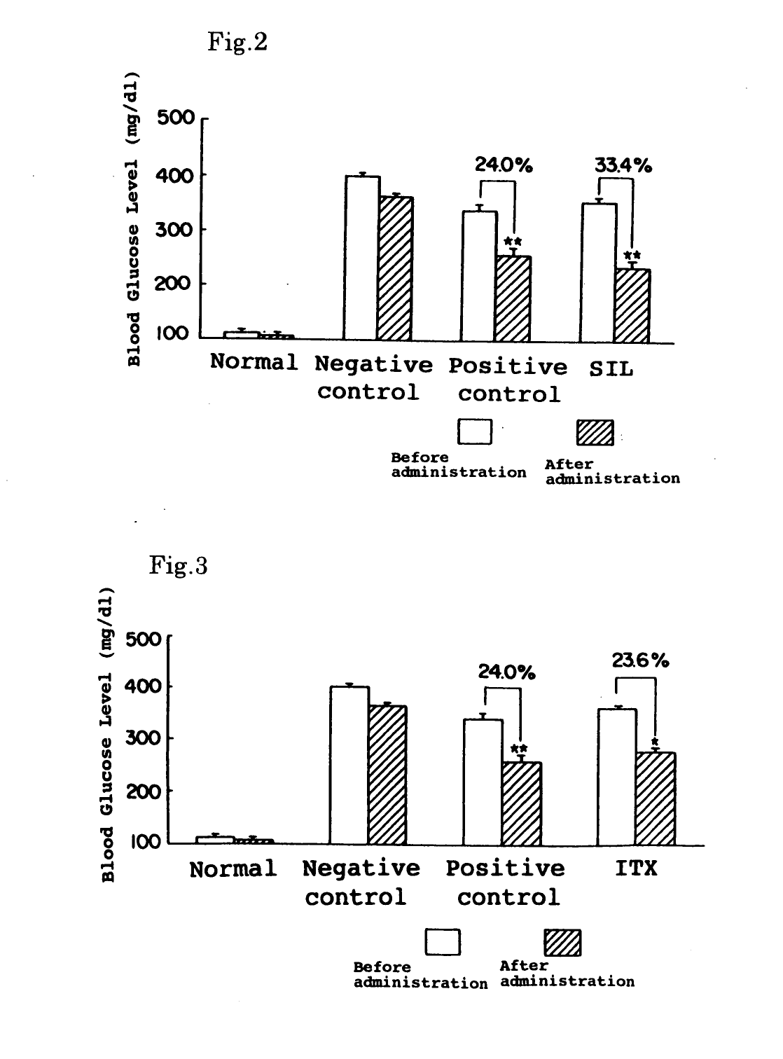

FIG. 2 shows the results of tests of SIL. Each bar shows average and standard deviation score, with asterisks (* mark) showing Student's t-test results (*p<0.05 **p<0.01, significantly different from control). The presentation of graphs and t-test results applies to FIGS. 3 to 10 as well.

SIL reduced the blood glucose level by 33.4 percent, indicating an effect similar to that of the positive control attaining 24.0 percent decrease in the blood glucose level.

FIG. 3 shows the results of tests of ITX. ITX reduced the blood glucose level by 23.6 percent, indicating an effect similar to that of the positive control attaining 24.0 percent reduction in the blood glucose level.

Table 2 shows the results of tests of TAX and hongdoushan fraction. TABLE-US-00002 TABLE 2 Effects of TAX and hongdoushan fractions on reduction in blood glucose level of diabetic rats induced by injecting STZ Blood glucose level (mg/dL) Before After Reduction Group administration administration (%) Normal 111.3.+−.13.2 106.1.+−.12.7 Negative control 402.3.+−.11.4 364.4.+−.13.0 Positive control 339.8.+−.23.5 258.2.+−.27.6 24 TAX 316.1.+−.19.9 250.2.+−.35.5 20.9 Aqueous extract 385.7.+−.12.8 255.6.+−.39.1 33.7 Methanol extract 328.1.+−.10.0 350.6.+−.25.0-6.9 Ethyl acetate soluble 419.2.+−.10.7 368.2.+−.10.4 12.1 fraction Ethyl acetate insoluble 449.6.+−.36.2 392.8.+−.19.6 12.6 fraction

TAX reduced the blood glucose level by 20.9 percent, indicating an effect similar to that of the positive control attaining 24.0 percent reduction in the blood glucose level.

Ethyl acetate soluble fraction reduced the blood glucose level by 12.1 percent.

### Example 2

### Hepatoprotective Activity

Activities of compounds of lignans and hongdoushan fractions in prevention and remedy of hepatic damages were assessed by means of the following method:

Evaluation by D-galactosamine (hereinafter called “D-GalN”)/Lipopolysaccharide (hereinafter called “LPS”)-induced hepatic damage model (J. Wang et al., Biochem. Pharm., 39,267 (1990), A. Wendel et al., Biochem. Pharm., 35, 2115 (1986))

Male mice of ddY strains (age: 6 weeks) kept fast for 12 hours, and then hepatic damage was induced in the mice by injecting D-GalN (700 mg/kg)/LPS (10.mu.g/kg) into their abdominal cavity. Test compounds were given subcutaneously to the mice 2 times: 12 hours and 1 hour before the injection of D-GalN/LPS. They were arranged into 2 groups which were given test compounds: a group in a dose of 50 mg/kg (weight of rat), and a group in a dose of 10 mg/kg (weight of rat). There were also a control group which was given normal saline solution and a group which was given silymarin, a known hepatoprotective agent, (subcutaneous administration in a dose of 100 mg/kg) for comparison of medicinal benefits.

Tumor necrosis factor alpha (hereinafter called “TNF-.alpha.”) level in the blood was measured 90 minutes after the injection of D-GalN/LPS. Furthermore, levels of GPT (glutamic-pyruvic transaminase) and GOT (glutamic oxaloacetic transaminase) in the blood were measured 8 hours after the injection.

TNF-.alpha. level was measured by means of the ELISA method using an anti-mouse TNF-.alpha. antibody (manufactured by Endogen, Inc., USA), and levels of GPT and GOT were measured by using a Transaminase CII-Test kit (manufactured by Wako Pure Chemical Industries, Ltd.).

The hepatic damage mechanism induced in this damage model is thought to have high correlativity with clinical results as a model of immunological hepatic damage outbreak because of a series of process such as activation of cells involved in immunity, infiltration into hepatic tissues, secretion of cytokine and autocoid such as leukotriene D4 and TNF-.alpha., and apoptosis of hepatic cells.

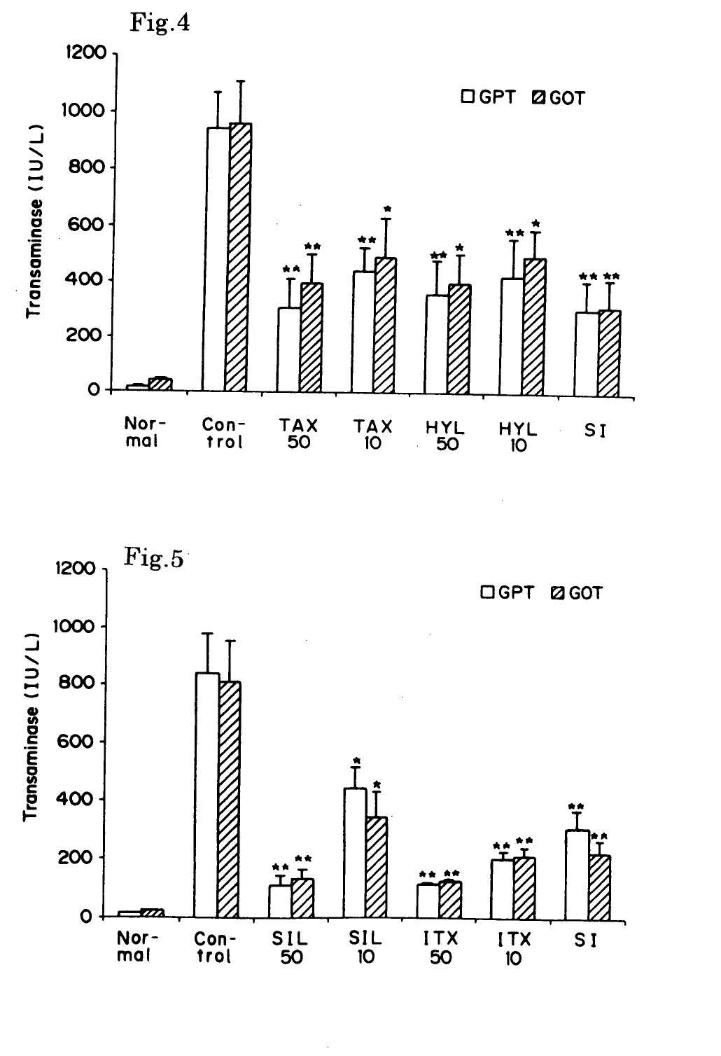

FIGS. 4 and 5 show the results of measurement of transaminase levels. The vertical axis of the graph represents transaminase level in blood serum in IU/L. Normal is a group which was given no D-GalN/LPS. Control is a group which was given normal saline solution. TAX 50-ITX 10 are the names of compounds of lignans and doses thereof. SI is a group which was given silymarin. The normal group had 3 mice, and the other groups had 6 mice each.

The results show that TAX, HYL, SIL, and ITX in a dose of 10 mg/kg and 50 mg/kg inhibit the increase in GPT and GOT levels in blood serum, hence indicating significant hepatoprotective effects in a dose-dependent manner.

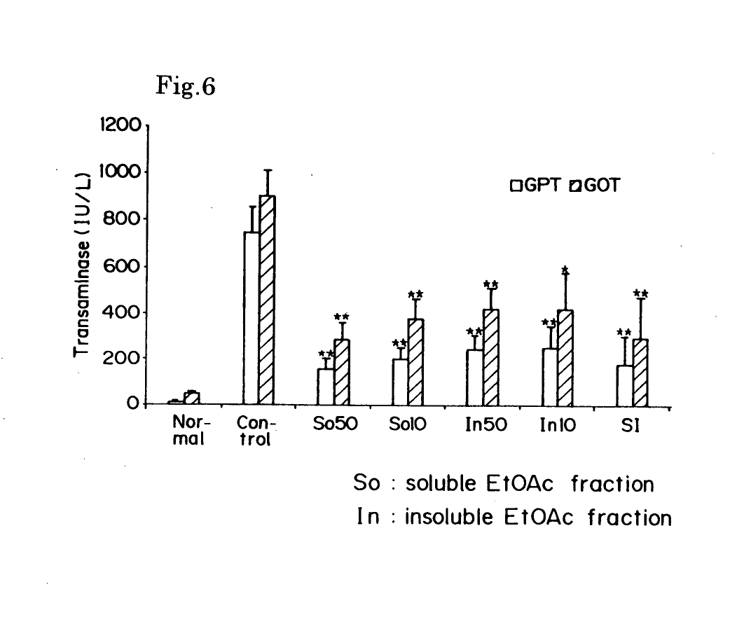

FIG. 6 shows the results of measurement of transaminase level in the case of ethyl acetate soluble fraction of hongdoushan being given (“So” in the graph). Like compounds of lignans, ethyl acetate soluble fraction inhibits the increase in GPT and GOT levels in blood serum, hence indicating significant hepatoprotective effects in dose-dependent manner.

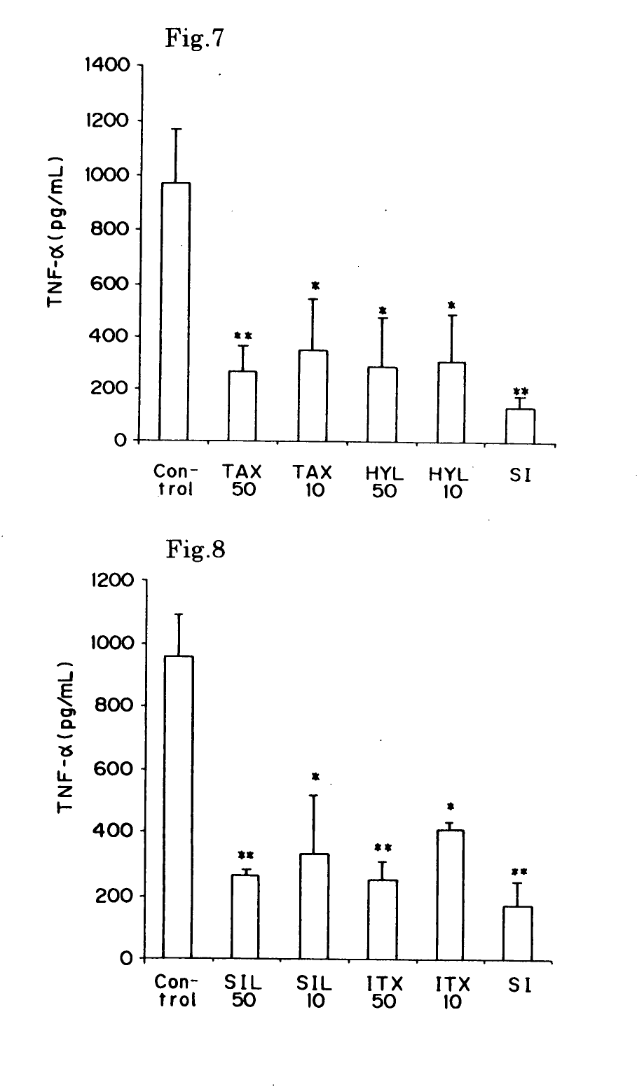

FIGS. 7 and 8 show the results of measurement of TNF-.alpha. The vertical axis of the graph represents TNF-.alpha. level in blood serum in pg/mL. Control is a group which was given normal saline solution. TAX 50-ITX 10 are the names of compounds of lignans and doses thereof. SI is a group which was given silymarin. Each group had 6 rats. The TNF-.alpha. level in blood serum of a D-GalN/LPS-untreated group (3 rats) is not shown in the figures because of the level being below measurable limit (10 pg/mL).

The results show that TAX, HYL, SIL, and ITX in a dose of 10 mg/kg and 50 mg/kg inhibit the increase in TNF-.alpha. level in blood serum, hence indicating significant hepatoprotective effects in dose-dependent manner.

Hepatic cells of the mice which were given SIL and ITX and of the mice of the control group were histopathologically observed 8 hours after the injection of D-GalN/LPS. The observation of the mice of the control group showed that there were many apoptosis corpuscles in the cells and that there were many concentrated chromatins in the cell nucleuses. This means that many apoptotic cells were observed. On the other hand, the observation of the mice which were given SIL and ITX showed less apoptosis corpuscles in the cells and less concentrated chromatins in the cell nucleuses. A series of the histopathological observations also supports effectiveness of compounds of lignans in inhibiting hepatic damages.

### Example 3

### Inhibitory Activity on TNF-.alpha.-Induced Cell Death in Primary Cultured Mouse Hepatocytes

Hepatic parenchymal cells were separated from the lives of male mice of ddY strains by means of a collagenase recirculation method. The separated hepatic parenchymal cells were suspended in the William's E culture medium, which was supplemented with 10% calf blood serum, 100 IU/mL penicillin G, 100 g/mL streptomycin, 100.mu.M dexamethasone, and 50 ng/mL insulin, and incubated in a 96-well plastic plate (1.5.times.10.sup.4 cells/well). The culture medium was replaced with a fresh culture medium containing D-galactosamine (0.5 mM) and lignans after 2-hour pre-incubation. TNF-.alpha. (100 ng/mL) was added to each well 30 minutes later. The hepatocytes viability was assessed 18 hours later by means of MTT(3-(4,5-dimethylthiazol-2-yl)-2,5-dimethyltetrazolium bromide) colorimetric reaction.

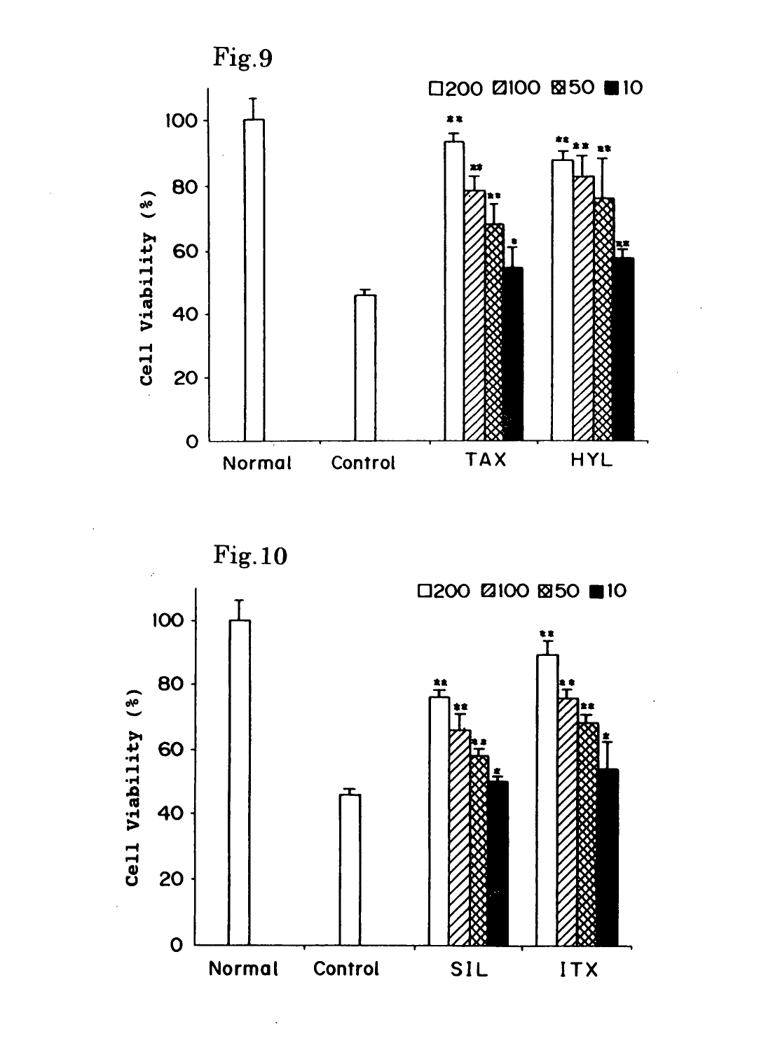

FIGS. 9 and 10 show the results of the assessment.

The vertical axis of the graph represents cell viability in percentage. The bar of Normal is cell viability in the TNF-.alpha.-untreated culture medium. The bar of Control is cell viability in the test compound-untreated culture medium.

TAX, HYL, SIL, and ITX were treated at 200.mu.M, 100.mu.M, 50.mu.M, and 10.mu.M in the culture medium. Cell viability of each item is shown in the bar graphs.

The treatment of TAX in the culture medium increased cell viability considerably in a dose-dependent manner, compared with Control. The treatment of HYL, SIL, and ITX showed the results similar to that of TAX.

### Example 4

### Antiproliferative Activity Against Cultured Cancer Cells

Compounds of lignans and hongdoushan fractions were tested for their antiproliferative activity against human HT-1080 fibrosarcoma cells and murine colon 26-L5 carcinoma cells. Human HT-1080 fibrosarcoma cells were cultured in the EMEM medium supplemented with 10% FCS (heat-inactivated fetal calf serum) and 0.1% sodium bicarbonate, and 2 mM glutamine. On the other hand, murine colon 26-L5 carcinoma cells were cultured in the RPMI medium containing the same supplements as in the EMEM medium. Cell viability was determined by means of MTT(3-(4,5-dimethylthiazol-2-yl)-2,5-dimethyltetrazolium bromide).

The cells were cultured in a 96-well plate, which was supplemented with a cell suspension medium solution. In this case, each well had approximately 2,000 cells in 100.mu.L. The cells grew exponentially during the cultivation.

Twenty four hours later, when the cells adhered to the plate, the medium solution was removed, and then the cells were treated with a test compound medium solution (100.mu.L) at various concentrations (100.mu.g/mL, 50.mu.g/mL, 10.mu.g/mL, 5.mu.g/mL, and 1.mu.g/mL). The cells were cultured at 37.degree.C. under 5% CO.sub.2. In this case, the test compounds were first dissolved in DMSO and then diluted in the medium. The concentration of DMSO was adjusted to be finally below 0.25%.

Next, the medium solution was removed, and the dissolved MTT (100.mu.L) was added to the medium. After three-hour incubation of the cells, the amount of the formazan formed was measured spectrophotometrically at 550 nm with a plate reader HTS-7000 (manufactured by Perkin Elmer). The measurements at one concentration were carried out in 4 wells, resulting in an IC.sub.50 ((50% effective concentration) value of each compound calculated from the average of 4 measurements.

Tables 3 and 4 show the results of the measurements. TABLE-US-00003 TABLE 3 Cell Antiproliferative activity of lignans compounds IC.sub.50 (.mu.g/mL) Compounds Colon 26-L5 HT-1080 TAX 35.2 62.2 HYL>100>100 SIL 60.2 5.9 ITX 36.5 43.8 5-Fluorouracil 0.07 0.29

SIL showed noticeable antiproliferative activity against HT-1080 cells. TAX and ITX showed antiproliferative activity against both HT-1080 and Colon 26-L5. TABLE-US-00004 TABLE 4 Cell Antiproliferative activity of lignans compounds IC.sub.50 (.mu.g/mL) Colon Fractions 26-L5 HT-1080 Aqueous extraction 69.5 9.5 Methanol/Aqueous 8.2<1.0 extraction Methanol extraction 15.3<1.0 Ethyl acetate soluble 8.7 2.6 fraction Ethyl acetate 9.2 8.2 insoluble fraction

Ethyl acetate soluble fraction showed antiproliferative activity against both HT-1080 and Colon 26-L5.

### Example 5

### DPPH Free Radical Scavenging Activity

500.mu.L of each test compound dissolved in ethanol or aqueous solution was mixed with 500.mu.L of DPPH (1,1-diphenyl 1-2-picrylhydrazyl)ethanol solution (concentration: 60.mu.M). The mixtures were left at room temperature for 30 minutes, and then their absorbance was measured at 520 nm. The measurements were carried out for each test compound at different concentrations. EC.sub.50 (50% effective concentration) values were calculated from the measurement results.

Table 5 shows the results of the measurements. TABLE-US-00005 TABLE 5 DPPH free radical scavenging activity of hongdoushan fractions Fractions ED.sub.50 (.mu.g/mL) Aqueous extract 8.7 Methanol/Aqueous 8.3 extract Methanol extract 9.9 Ethyl acetate soluble 9.5 fraction Ethyl acetate 18.1 insoluble fraction Caffeic Acid 5.41

Ethyl acetate soluble fraction showed noticeable DPPH free radical scavenging activity.

DPPH free radical scavenging activity is one antioxidant activity. Substances with strong free radical scavenging activity are thought to have strong antioxidant activity. Therefore, an action mechanism of ethyl acetate soluble fraction against D-GalN/TNF-.alpha.-induced hepatic damage is thought to lie in antioxidant activity which inhibits production of TNF-.alpha., hence inhibiting apoptosis of hepatic cells.

It has been reported that antioxidant substances are effective for remedy of complications such as cataract caused by diabetes. Therefore, ethyl acetate soluble fraction and lignans of the present invention are thought to be effective for remedy of diabetes complications as well.

### INDUSTRIAL APPLICABILITY

Compounds and hongdoushan organic solvent extract fractions of the present invention are useful as drugs, in particular, suitable for hypoglycemic agent, hepatoprotective agent or anticancer agent.

We claim:

1. A method of reducing the blood glucose level of a patient comprising the step of:
administering to the patient a compound shown in the formula (1) 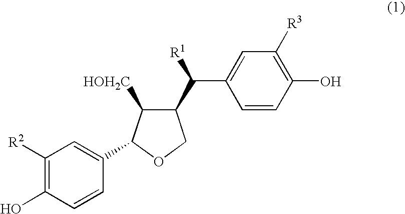
(wherein R¹ is a hydrogen or a hydroxyl group, R² is either a hydroxyl group or an alkyloxy group with a carbon number of 1 to 4, and R³ is an alkyloxy group with a carbon number of 1 to 4) or a pharmaceutically acceptable salt or ester of said compound in the formula (1) as an effective ingredient.

2. A method of eliciting a hepatoprotective effect comprising the step of:
treating a patient with a compound shown in the formula (1) 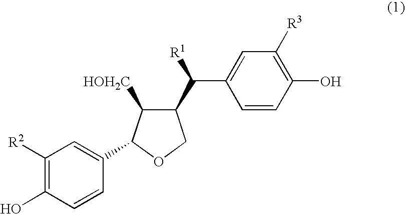
(wherein R¹ is a hydrogen or a hydroxyl group, R² is either a hydroxyl group or an alkyloxy group with a carbon number of 1 to 4, and R³ is an alkyloxy group with a carbon number of 1 to 4) or a pharmaceutically acceptable salt or ester of said compound in the formula (1) as an effective ingredient.

3. The method of claim 1, wherein said hepatoprotective effect is an inhibition of an increase in levels of glutamic-pyruvic transaminase and glutamic oxaloacetic transaminase in bloom serum.
